(19) United States
(12) Patent Application Publication
(10) Pub. No.: US 2020/0170904 A1
(43) Pub. Date: Jun. 4, 2020

(54) HAIR COLORING COMPOSITION WITH IMPROVED SALT TOLERANCE

(71) Applicant: Henkel AG & Co. KGaA, Duesseldorf (DE)

(72) Inventors: BURKHARD MUELLER, Duesseldorf (DE); LUCILE BONNIN, Berlin (DE)

(73) Assignee: Henkel AG & Co. KGaA, Duesseldorf (DE)

(21) Appl. No.: 16/620,824

(22) PCT Filed: May 14, 2018
(86) PCT No.: PCT/EP2018/062290

§ 371 Date: Dec. 9, 2019

(30) Foreign Application Priority Data
Jun. 9, 2017 (DE) 10 2017 209 769.8

(51) Int. Cl. A61K 8/34; A61Q 5/10; A61K 8/37; A61K 8/86; A61K 8/39; A61K 8/22; A61K 8/46

(52) U.S. Cl. CPC A61K 8/342; A61Q 5/10; A61K 8/37; A61K 8/86; A61K 2800/4324; A61K 8/22; A61K 8/463; A61K 2800/87; A61K 2800/882; A61K 8/39

(57) ABSTRACT

A composition for dyeing keratinous fibres comprises, in an aqueous carrier, (a) highly ethoxylated C₁₆-C₁₈ fatty alcohol(s) with from about 30 to about 100 ethoxy groups, (b) low-ethoxylated C₁₆-C₁₈ fatty alcohol(s) with 1 to 5 ethoxy groups, (c) optionally monoester(s) of glycerol and a C₁₄-C₂₀ fatty acid, (d) C₁₆-C₁₈ fatty alcohol(s), (e) alkalising agent(s), and (f) oxidation dye precursor(s), wherein—the weight ratio of all highly ethoxylated C₁₆-C₁₈ fatty alcohols (a) to all low-ethoxylated C₁₆-C₁₈ fatty alcohols (b) is from about 10 to about 20, and the weight ratio of the sum of all constituents [(c)+(d)] to the sum of all ethoxylated fatty alcohols [(a)+(b)] is from about 2 to about 5. The present disclosure further relates to a multi-component packaging unit, in which the aforementioned composition is packaged separately from an oxidant preparation.

### CROSS-REFERENCE TO RELATED APPLICATION

This application is a U.S. National-Stage entry under 35 U.S.C. § 371 based on International Application No. PCT/EP2018/062290, filed May 14, 2018, which was published under PCT Article 21 (2) and which claims priority to German Application No. 10 2017 209 769.8, filed Jun. 9, 2017, which are all hereby incorporated in their entirety by reference.

### TECHNICAL FIELD

The present disclosure lies in the field of cosmetics. The present disclosure relates to compositions for dyeing keratinous fibres which contain not only dyes and alkalising agents but also a specific combination of (a) highly ethoxylated fatty alcohols, (b) low-ethoxylated fatty alcohols, optionally (c) monoglycerol fatty acid esters, and (d) fatty alcohols. A characterising feature of these compositions is that they contain the aforementioned ingredients (a) to (d) in quite specific ratios by weight.

The present disclosure further relates to a multi-component packaging unit, in which the aforementioned agents are provided separately in addition to an oxidant preparation.

### BACKGROUND

The use of emulsions is widespread in cosmetics. An emulsion comprises a finely dispersed mixture of two liquids, such as fat bodies (oils, fatty alcohols, hydrocarbons or also fatty acid triglycerides) and water. One theory regarding emulsions is that one of the liquids (phase) forms small droplets which are present dispersed in the other liquid (phase). The phase that forms the droplets is referred to as the inner phase or also disperse phase. The phase in which the droplets float is called the outer phase or also the continuous phase.

In the case of emulsions that comprise a water phase and an oil phase, a distinction is made between oil-in-water emulsions (O/W emulsions) and water-in-oil emulsions (W/O emulsions). Classic O/W emulsions are described in the literature often as oil droplets which are dispersed in the continuous water phase and are stabilised at the interface of both phases by surfactants or emulsifiers. The latter form a film around the oil droplets and are thus able to reduce the surface tension. In complex cosmetic formulations, however, a number of various ingredients are generally used, thus resulting in complex multiphase systems.

Many compositions for colouring keratinous fibres or human hair are present in the form of emulsions. An emulsion of this kind may then be referred to as stable if the fusion of the droplets can be prevented by a sufficiently high energy barrier. Generally, this energy barrier is formed by the film forming the one or more emulsifiers on the surface of the particular droplet. If the emulsion is unstable, it breaks and separates into the oil and water phase. Cosmetic products, in particular hair colouring compositions, often have to endure storage times of many months. It is an essential quality requirement that the colouring compositions in emulsion form remain stable over the entire storage period and do not separate during the storage.

In order to produce permanent, intense colourations with corresponding fastness properties, what are known as oxidation dyes are used. Such dyes usually contain oxidation dye precursors, or what are known as developer components and coupler components. The developer components either themselves or with coupling with one or more coupler components form, under the influence of oxidants or atmospheric oxygen, the actual dyes per se. The oxidation dyes are exemplified by intense, outstanding, long-lasting colour results. A mixture of a greater number of oxidation dye precursors can be used for naturally acting dyeing agents, wherein substantive dyes are also used additionally in many cases to provide further colour shades.

Oxidation dye precursors of the developer type are based typically on the basic structure of p-phenylenediamine, p-aminophenol, or heterocyclic di- or polyamino compounds. Substances of this type are extremely sensitive to atmospheric oxygen and are usually used in the form of their physiologically acceptable salts for stabilisation, i.e. the amino groups provided in the substances are converted—wholly or partially—into ammonium groups and are neutralised by counterions (chlorides, bromides, hydrogen sulfates or also sulfates). If a user wishes to colour their hair in a particularly dark shade, for example a dark-brown or black shade, said user will thus use a corresponding colouring composition with a particularly high dye content. Due to the high content of oxidation dye precursors, the corresponding salt content in these compositions is also very high.

Emulsions, such as O/W emulsions, often react very sensitively to an increase of their salt content. The risk that an emulsion or a colouring composition will separate and prove unstable under storage is therefore particularly high in the case of shades having a high dye content.

Furthermore, the commercial distribution of a hair dye brand generally includes a colour shade portfolio from which the user can choose his/her desired colour.

Within this portfolio, a base cream that is the same for all shades is usually used, in which base cream different quantities of oxidation dye precursors are used depending on the particular shade. The darker is the shade, the higher is the content of oxidation dyes and the higher is thus also the salt content in the emulsion. So as to be able to use the same base cream within the portfolio, it is of central importance that this cream has a uniform and high stability, both with use of low and with use of high dye concentrations. In order to simplify the production processes, the viscosity of the dye cream should also remain the same, regardless of the selected dye quantity.

### BRIEF SUMMARY

A composition for dyeing keratinous fibres and a multi-component packaging unit (kit of parts) are provided. In an exemplary embodiment, the composition for dyeing keratinous fibres comprises, in an aqueous carrier, (a) one or more highly ethoxylated C₁₆-C₁₈ fatty alcohols with from about 30 to about 100 ethoxy groups, (b) one or more low-ethoxylated C₁₆-C₁₈ fatty alcohols with from about 1 to about 5 ethoxy groups, (c) optionally one or more monoesters of glycerol and a C₁₄-C₂₀ fatty acid, (d) one or more C₁₆-C₁₈ fatty alcohols, (e) one or more alkalising agents, and (f) one or more oxidation dye precursors, wherein the weight ratio of all highly ethoxylated C₁₆-C₁₈ fatty alcohols (a) to all low-ethoxylated C₁₆-C₁₈ fatty alcohols (b) is from about 10 to about 20, and the weight ratio of the sum of all constituents [(c)+(d)] to the sum of all ethoxylated fatty alcohols [(a)+(b)] is from about 2 to about 5.

An exemplary multi-component packaging unit (kit-of-parts) for the oxidative dyeing of keratin fibres, comprises, packaged separately from one another, —a container (I) comprising a cosmetic agent (A), and —a container (II) comprising a cosmetic agent (B), wherein the agent (A) is the composition for dyeing keratinous fibres of claim 1, and the agent (B) is an oxidant preparation comprising hydrogen peroxide.

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The object of the present disclosure was therefore to provide colouring compositions (in particular dye creams for oxidative colour change) in emulsion form which have an improved stability with increased salt tolerance and optimised viscosity stability. Since the colour creams are generally set to be alkaline, this stabilisation should be ensured also under alkaline conditions.

The fundamental precondition here was that the colouring compositions should demonstrate the above-mentioned improvements without suffering any losses in respect of the further application properties. The colour intensity, washing fastness, and light fastness of these compositions therefore should not deteriorate compared to the compositions known from the prior art and, optimally, should be improved further.

It has now surprisingly been found that the salt tolerance of an alkaline dye cream was able to be significantly improved if a quite specific combination of non-ionic, lipophilic and hydrophilic emulsifiers was used in certain quantity ranges relative to one another. Particularly stable emulsions insensitive to fluctuating salt quantities were thus obtained when at least one highly ethoxylated C₁₆-C₁₈ fatty alcohol (with from about 30 to about 100 EO groups), (b) at least one low-ethoxylated C₁₆-C₁₈ fatty alcohol 9 with 1 to 5 EO groups), (c) optionally at least one monoester from glycerol and a C₁₄-C₂₀ fatty acid, and (d) at least one C₁₆-C₁₈ fatty alcohol were used in the emulsions (a).

Here, the weight ratios in which the constituents (a) to (d) are present in the alkaline dye cream have proven to be essential. A first important precondition is thus to use the highly ethoxylated C₁₆-C₁₈ fatty alcohols (a) in a from about 10-fold to about 20-fold weight excess in the composition in comparison to the low-ethoxylated C₁₆-C₁₈ fatty alcohols (b), i.e. the weight ratio (a)/(b) lies at a value of from about 10 to about 20 in the composition as contemplated herein.

A second important precondition lies in the fact that the lipophilic emulsifier constituents (c) plus (d) are likewise used in excess in comparison to the ethoxylated fatty alcohols (a) plus (b). Here, the sum of the weight quantities of [(c)+(d)] used in the composition is from about 2 to about 5 times higher in comparison to the sum of the weight quantities of [(a)+(b)] used in the composition.

A first subject of the present disclosure is a composition for dyeing keratinous fibres, containing in an aqueous carrier
(a) one or more highly ethoxylated C16-C18 fatty alcohols with from about 30 to about 100 ethoxy groups,
(b) one or more low-ethoxylated C16-C18 fatty alcohols with 1 to 5 ethoxy groups,
(c) optionally one or more monoesters of glycerol and a C14-C20 fatty acid,
(d) one or more C16-C18 fatty alcohols,
(e) one or more alkalising agents, and
(f) one or more oxidation dye precursors, wherein
   the weight ratio of all highly ethoxylated C₁₆-C₁₈ fatty alcohols (a) contained in the composition to all low-ethoxylated C₁₆-C₁₈ fatty alcohols (b) contained in the composition, i.e. the weight ratio (a)/(b), lies at a value of from about 10 to about 20, and
   the weight ratio of the sum of all constituents [(c)+(d)] contained in the composition to the sum of all ethoxylated fatty alcohols [(a)+(b)] contained in the composition, i.e. the weight ratio [(c)+(d)]/[(a)+(b)], lies at a value of from about 2 to about 5.

### Keratinous Fibres

The composition is a composition for dyeing keratinous fibres, in particular human hair. The composition is particularly preferably used for the oxidative colouring of keratinous fibres, in particular human hair.

Keratinous fibres are understood to mean wools, furs, feathers and in particular human hair. The compositions as contemplated herein for oxidative changing of colour, however, can also be used in principle for changing the colour of other natural fibres, such as cotton, jute, sisal, flax or silk, modified natural fibres, such as regenerated cellulose, or nitro-, alkyl- or hydroxyalkyl- or acetylcellulose.

### Composition for Dyeing Keratinous Fibres

The composition as contemplated herein is used for the dyeing, in particular for the oxidative dyeing, of keratinous fibres. With the constituents (e) and (f), the composition contains alkalising agents and oxidation dye precursors. The composition is thus an alkaline dye cream which is mixed with an oxidant before use and is then applied as a ready-to-use composition to the keratin fibres (hair).

The composition contains, as constituents essential to the present disclosure, the components (a) and (b) and (d) and (e) and (f). The component (c) can furthermore be contained as an optional constituent in the composition.

### Aqueous Carrier

The composition as contemplated herein contains the constituents (a) to (f) (wherein the constituent (c) is optional) in an aqueous carrier. The constituents (a) and (b) represent fatty alcohols with a high or low degree of ethoxylation. The optional constituent (c) is a fatty acid monoglyceride, and the constituent (d) is constituted by C₁₆-C₁₈ fatty alcohols. Thus, (a) to (d) are non-ionic compounds with emulsifier properties which differ in respect of their lipophilicity or hydrophilicity. Together, the constituents form an emulsion which is an oil-in-water emulsion. Here, water is the outer phase or also the continuous phase.

The composition as contemplated herein particularly preferably contains—in relation to its total weight—from about 50 to about 95% by weight, preferably from about 55 to about 90% by weight, more preferably from about 60 to about 85% by weight, and very particularly preferably from about 65 to about 80% by weight of water.

### (a) Highly Ethoxylated Fatty Alcohols

As first constituent (a) essential to the present disclosure, the compositions as contemplated herein contain one or more highly ethoxylated C₁₆-C₁₈ fatty alcohols with from about 30 to about 100 ethoxy groups. In this case the structural unit —CH2—CH2—O—, which can also be referred to alternatively as an ethylene oxide unit, is referred to as ethoxy group.

The amount of substance of ethylene oxide that was used per mole of fatty alcohol, refers in this case to the degree of ethoxylation. Highly ethoxylated fatty alcohols are therefore understood to mean fatty alcohols which were ethoxylated with at least about 30 and at most about 100 ethoxy groups (i.e. each mole of fatty alcohol was ethoxylated with from about 30 mol to about 100 mol ethylene oxide).

In other words a composition as contemplated herein contains (a) one or more ethoxylated C₁₆-C₁₈ fatty alcohols with a degree of ethoxylation of from about 30 to about 100.

C₁₆-C₁₈ fatty alcohols as contemplated herein are linear or branched, saturated or unsaturated alkanols with 16 to 18 carbon atoms. Unsaturated C₁₆-C₁₈ fatty alcohols can be mono- or polyunsaturated. In the case of an unsaturated fatty alcohol the C—C double bond(s) thereof can have the cis or trans configuration. It is likewise possible as contemplated herein to use mixtures of fatty alcohols which accrue by selective mixing or also by extraction processes as such. One example is cetearyl alcohol (about 1:1 mixture of C₁₆ and C₁₈ fatty alcohols).

The composition particularly preferably contains, as highly ethoxylated C₁₆-C₁₈ fatty alcohol, at least one compound of formula (I)

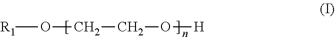

in which
R1 represents a linear or branched, saturated or unsaturated C16-C18 alkyl group, particularly preferably for a cetyl group (C16 alkyl group) or a stearyl group (C18 alkyl group) and
n represents an integer from about 30 to about 100, preferably from about 35 to about 90, more preferably from about 40 to about 80, and very particularly preferably from about 45 to about 70.

In a particularly preferred embodiment a composition as contemplated herein contains (a) one or more highly ethoxylated C₁₆-C₁₈ fatty alcohols of formula (I),

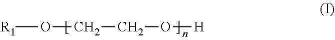

in which
R1 represents a linear or branched, saturated or unsaturated C16-C18 alkyl group and
n represents an integer from about 30 to about 100, preferably from about 35 to about 90, more preferably from about 40 to about 80, and very particularly preferably from about 45 to about 70.

Suitable highly ethoxylated fatty alcohols of formula (I) include, for example, Ceteareth-30, Steareth-30, Ceteth-30, Oleth-30, Ceteareth-50, Steareth-50, Ceteth-50, Oleth-50, Ceteareth-100, Steareth-100, Ceteth-100 and Oleth-100.

Ceteareth-50, Steareth-50, and/or Ceteth-50 are very particularly preferably used in the compositions as contemplated herein.

The highly ethoxylated C₁₆-C₁₈ fatty alcohols are preferably used in the composition as contemplated herein in specific quantity ranges, wherein for all quantity ranges (a) the premise applies that the two ratio conditions regarding the weight ratios (a)/(b) and [(c)+(d)]/[(a)+(b)] are satisfied.

Particularly stable and salt-tolerant emulsions could be obtained if the compositions contained one or more highly ethoxylated C₁₆-C₁₈ fatty alcohols with from about 30 to about 100 ethoxy groups in a total quantity of from about 2.0 to about 10.0% by weight, preferably from about 2.0 to about 8.0% by weight, more preferably from about 2.0 to about 6.0% by weight, and very particularly preferably from about 3.0 to about 5.0% by weight. All aforementioned quantity ranges in % by weight are understood here to relate to the total quantity of the highly ethoxylated fatty alcohols (a) contained in the composition, which is set in relation to the total weight of the composition.

In a further very particularly preferred embodiment a composition as contemplated herein contains—in relation to the total weight of the composition—
(a) one or more highly ethoxylated C₁₆-C₁₈ fatty alcohols with from about 30 to about 100 ethoxy groups in a total quantity of from about 2.0 to about 10.0% by weight, preferably from about 2.0 to about 8.0% by weight, more preferably from about 2.0 to about 6.0% by weight, and very particularly preferably from about 3.0 to about 5.0% by weight.

### (b) Low-ethoxylated Fatty Alcohols

As second constituent (b) essential to the present disclosure, the compositions as contemplated herein contain one or more low-ethoxylated C₁₆-C₁₈ fatty alcohols with 1 to 5 ethoxy groups.

Low-ethoxylated fatty alcohols are understood to be fatty alcohols which have been ethoxylated with at least 1 and at most 5 ethoxy groups (EO groups). In this case the structural unit —CH2—CH2—O—, which can also be referred to alternatively as an ethylene oxide unit, is again referred to as ethoxy group.

In other words a composition as contemplated herein contains (b) one or more ethoxylated C₁₆-C₁₈ fatty alcohols with a degree of ethoxylation of from 1 to 5.

As described before, the C₁₆-C₁₈ fatty alcohols as contemplated herein are linear or branched, saturated or unsaturated alkanols with 16 to 18 carbon atoms. Unsaturated C₁₆-C₁₈ fatty alcohols can be mono- or polyunsaturated. In the case of an unsaturated fatty alcohol the C—C double bond(s) thereof can have the cis or trans configuration. It is likewise possible as contemplated herein to use mixtures of fatty alcohols which accrue by selective mixing or also by extraction processes as such. One example is cetearyl alcohol (about 1:1 mixture of C₁₆ and C₁₈ fatty alcohols).

The composition particularly preferably contains, as low-ethoxylated C₁₆-C₁₈ fatty alcohol (b), at least one compound of formula (II)

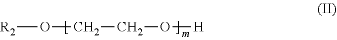

in which
R2 represents a linear or branched, saturated or unsaturated C16-C18 alkyl group and
M represents an integer from about 1 to about 5, preferably from about 1 to about 4, and very particularly preferably from about 1 to about 3.

In a particularly preferred embodiment a composition as contemplated herein contains (b) one or more low-ethoxylated C₁₆-C₁₈ fatty alcohols of formula (II),

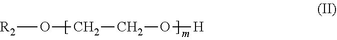

in which
R2 represents a linear or branched, saturated or unsaturated C16-C18 alkyl group and
M represents an integer from about 1 to about 5, preferably from about 1 to about 4, and very particularly preferably from about 1 to about 3.

Suitable low-ethoxylated fatty alcohols of formula (II) include, for example, Ceteareth-2, Steareth-2, Ceteth-2, Oleth-2, Ceteareth-3, Steareth-3, Ceteth-3, Oleth-3, Ceteareth-4, Steareth-4, Ceteth-4, Oleth-4, Ceteareth-5, Steareth-5, Ceteth-5 and/or Oleth-5.

Ceteareth-2, Steareth-2, and/or Ceteth-2 are very particularly preferably used in the compositions as contemplated herein.

In order to achieve optimal results in respect of the salt stability, the low-ethoxylated fatty alcohols with 1 to 5 ethoxy groups are preferably also used in the composition as contemplated herein in specific quantity ranges.

Particularly stable and salt-tolerant emulsions could be obtained if the compositions contained one or more low-ethoxylated C₁₆-C₁₈ fatty alcohols (b) with 1 to 5 ethoxy groups in a total quantity of from about 0.05 to about 1.5% by weight, preferably from about 0.05 to about 12.5% by weight, more preferably from about 0.1 to about 1.0% by weight, and very particularly preferably from about 0.1 to about 0.75% by weight.

Also with these quantities regarding the total quantity contained in the composition of low-ethoxylated fatty alcohols (b), the precondition still applies that at the same time the two ratio conditions regarding the weight ratios (a)/(b) and [(c)+(d)]/[(a)+(b)] must be satisfied.

All aforementioned quantity ranges in % by weight are understood here to relate to the total quantity of the low-ethoxylated fatty alcohols (b) contained in the composition, which is set in relation to the total weight of the composition.

In a further very particularly preferred embodiment a composition as contemplated herein contains—in relation to the total weight of the composition—
(b) one or more low-ethoxylated C₁₆-C₁₈ fatty alcohols with 1 to 5 ethoxy groups in a total quantity of from about 0.05 to about 1.5% by weight, preferably from about 0.05 to about 1.25% by weight, more preferably from about 0.1 to about 1.0% by weight, and very particularly preferably from about 0.1 to about 0.75% by weight.
   Weight Ratio (a)/(b)

Regarding the total quantities of highly ethoxylated fatty alcohols (a) and low-ethoxylated fatty alcohols (b) used in the compositions as contemplated herein, two ratio conditions must be satisfied.

The first ratio condition is that the weight ratio of all highly ethoxylated C₁₆-C₁₈ fatty alcohols (a) contained in the composition to all low-ethoxylated C₁₆-C₁₈ fatty alcohols (b) contained in the composition, i.e. the weight ratio (a)/(b), lies at a value of from about 10 to about 20.

In other words, the highly ethoxylated fatty alcohols (a) must be used in the composition in a from about 10-fold to about 20-fol weight excess compared to the low-ethoxylated fatty alcohols (b).

Without being fixed to this theory, it is alleged that, due to the choice of these specific quantity ratios of (a) and (b) (together with the further constituents (c) and (d)) an emulsion system is formed in which the various emulsifiers (a) to (d) are present in particularly stable films or phases. The emulsion produced in this way has a particularly high tolerance to varying salt concentrations. The result of this is that the use of different quantities of oxidation dyes used in salt form has a particularly small effect on the rheological properties of the emulsion.

In this regard it has proven to be very particularly preferred if the weight ratio of all highly ethoxylated C₁₆-C₁₈ fatty alcohols (a) contained in the composition to all low-ethoxylated C₁₆-C₁₈ fatty alcohols (b) contained in the composition, i.e. the ratio weight (a)/(b), lies at a value of from about 11 to about 19, preferably from about 12 to about 18, more preferably from about 13 to about 17, and very particularly preferably from about 14 to about 16.

In a further very particularly preferred embodiment a composition as contemplated herein is thus exemplified in that the weight ratio of all highly ethoxylated C₁₆-C₁₈ fatty alcohols (a) contained in the composition to all low-ethoxylated C₁₆-C₁₈ fatty alcohols (b) contained in the composition, i.e. the weight ratio (a)/(b), lies at a value of from about 11 to about 19, preferably from about 12 to about 18, more preferably from about 13 to about 17, and very particularly preferably from about 14 to about 16.

Example: a dye cream contains, in addition to ammonia (e) and oxidation dyes (f),
(a) about 3.0% by weight Ceteth-50 and about 1.0% by weight Steareth-50 (total quantity of (a)=4.0% by weight) (b) about 0.2% by weight Ceteth-2 and about 0.2% by weight Steareth-3 (total quantity of (b)=about 0.4% by weight). The weight ratio (a)/(b) lies at about 4.0% by weight/about 0.4% by weight=about 10

Example: a dye cream contains, in addition to ethanolamine (e) and oxidation dyes (f),
(a) about 3.0% by weight Ceteth-50 and about 1.0% by weight Steareth-50 (total quantity of (a)=about 4.0% by weight) (b) about 0.1% by weight Ceteth-2 and about 0.1% by weight Steareth-3 (total quantity of (b)=about 0.2% by weight). The weight ratio (a)/(b) lies at about 4.0% by weight/about 0.2% by weight =about 20
   (c) Monoesters of Glycerol and C₁₄-C₂₀ Fatty Acid,

As optional constituent (c) the compositions as contemplated herein contain one or more monoesters of glycerol and a C₁₄-C₂₀ fatty acid.

Corresponding esters of group (c) are thus produced in each case by esterification of an equivalent (=mol equivalent) of glycerol and an equivalent (=mol equivalent) of C₁₄-C₂₀ fatty acid.

C₁₄-C₂₀ fatty acids, as contemplated herein, are understood to mean saturated or unsaturated, unbranched or branched, C₁₄-C₂₀ carboxylic acids. Unsaturated fatty acids can be mono- or polyunsaturated. In the case of an unsaturated fatty acid the C-C double bond(s) thereof can have the cis or trans configuration.

The fatty acid triglycerides are exemplified by particular suitability, wherein an ester group starting from glycerol is formed with a fatty acid selected from tetradecanoic acid (myristic acid), hexadecanoic acid (palmitic acid), tetracosanoic acid (lignoceric acid), octadecanoic acid (stearic acid), eicosanoic acid (arachinic acid), petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], linoleic acid [(9Z, 12Z)-octadeca-9,12-dienoic acid, linolenic acid [(9Z,12Z,15Z)-octadeca-9,12,15-trienoic acid, eleostearic acid [(9Z,11E,13E)-octadeca-9,11,3-trienoic acid], and/or arachidonic acid [(5Z,8Z,11Z,14Z)-icosa-5,8,11,14-tetraenic acid].

The composition as contemplated herein very particularly preferably contains (c) one or more monoesters from glycerol and a C₁₄-C₂₀ fatty acid of formula (III),

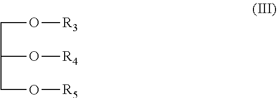

in which
R3, R4, R5, independently of one another, represent a hydrogen atom or an acyl group —C(O)R6, in which R6 represents an unbranched or branched, saturated or unsaturated C13-C19 alkyl group, preferably an unbranched, saturated C15-C17 alkyl group, with the provision that two of the groups from R3, R4 and R5 represent a hydrogen atom and the third group represents an acyl group —C(O)R6.

In a further very particularly preferred embodiment a composition as contemplated herein contains (c) one or more monoesters from glycerol and a C₁₄-C₂₀ fatty acid of formula (III),

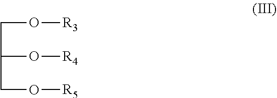

in which
R3, R4, R5, independently of one another, represent a hydrogen atom or an acyl group —C(O)R6, in which R6 represents an unbranched or branched, saturated or unsaturated C13-C19 alkyl group, preferably an unbranched, saturated C15-C17 alkyl group, with the provision that two of the groups from R3, R4 and R5 represent a hydrogen atom and the third group represents an acyl group —C(O)R6.

For example, one of the monoesters of glycerol and a C₁₄-C₂₀ fatty acid which is selected from the group of formulas (IIIa) to (IIId) can be used as a particularly well suited compound of formula (III).

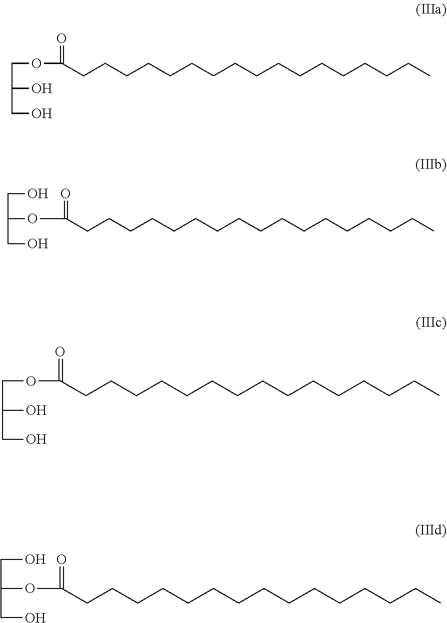 [SMILES: CCCCCCCCCCCCCCCCCC(=O)OCC(O)CO ; CCCCCCCCCCCCCCCCCC(=O)OC(CO)CO ; CCCCCCCCCCCCCCCC(=O)OCC(O)CO ; CCCCCCCCCCCCCCCC(=O)OC(CO)CO]

The compounds of formulas (IIIa) to (IIId) are also known under the names glyceryl monostearate and glycerylmonopalmitate.

The monoesters of glycerol and a C₁₄-C₂₀ fatty acid (c), together with the ingredients (a), (b) and (d), can form the emulsifier system as contemplated herein, which ensures a stable incorporation of the further constituents (e) and (f) (alkalising agents and oxidation dyes). The fatty acid monoglycerides (c) are therefore preferably also used in specific quantity ranges in the composition as contemplated herein.

Particularly stable and salt-tolerant emulsions could be obtained if the compositions contained one or more monoesters of glycerol and a C₁₄-C₂₀ fatty acid (c) in a total amount of from about 0.1 to about 5.0% by weight, preferably from about 0.1 to about 2.5% by weight, more preferably from about 0.1 to about 1.3% by weight, and very particularly preferably from about 0.2 to about 0.7% by weight.

The specified quantities in % by weight relate here again to the total quantity of all fatty acid monoglycerides (c) contained in the composition, which is set in relation to the total weight of the composition. Also with these quantities regarding the total quantity contained in the composition of monoester from glycerol and a C₁₄-C₂₀ fatty acid (c), the precondition still applies that in addition the ratio condition [(c)+(d)]/[(a)+(b)] must be satisfied.

Since (c) is an optional constituent, the quantity of (c) in the composition can also be zero. In order to satisfy the ratio condition [(c)+(d)]/[(a)+(b)] however, the quantity of (d) must then be selected to be higher, accordingly.

In a further very particularly preferred embodiment a composition as contemplated herein contains—in relation to the total weight of the composition—
(c) one or more monoesters from glycerol and a C₁₄-C₂₀ fatty acid in a total quantity from about 0.1 to about 5.0% by weight, preferably from about 0.1 to about 2.5% by weight, more preferably from about 0.1 to about 1.3% by weight, and very particularly preferably from about 0.2 to about 0.7% by weight.
   (d) C₁₆-C₁₈ is Fatty Alcohols

The presence of at least one C₁₆-C₁₈ fatty alcohol (d) is essential for forming the emulsion. The compositions as contemplated herein therefore contain one or more C₁₆-C₁₈ is fatty alcohols as essential ingredient (d). The C₁₆-C₁₈ fatty alcohols are saturated, mono- or polyunsaturated, linear or branched alkanols with 16 to 18 C atoms.

Examples of preferred linear, saturated C₁₆-C₁₈ fatty alcohols are hexadecan-1-ol (hexadecyl alcohol, cetyl alcohol, palmityl alcohol), octadecan-1-ol (octadecyl alcohol, stearyl alcohol) and mixtures thereof.

Preferred linear, unsaturated fatty alcohols are (9Z)-octadec-9-en-1-ol (oleyl alcohol), (9E)-octadec-9-en-1-ol (elaidyl alcohol), (9Z,12Z)-octadeca-9,12-dien-1-ol (linoleyl alcohol) and (9Z,12Z,15Z)-octadeca-9,12,15-trien-1-ol (linolenoyl alcohol).

In a particularly preferred embodiment the composition as contemplated herein contains at least one linear saturated C₁₆-C₁₈ fatty alcohol.

The C₁₆-C₁₈ fatty alcohols (d) in the dye creams as contemplated herein constitute the most lipophilic constituent of the emulsifier system including components (a) to (d). In order to optimise this emulsifier system, the one or more C₁₆-C₁₈ fatty alcohols (d) are also used in the composition as contemplated herein particularly preferably in specific quantity ranges. Also with these quantities regarding the total quantity of C₁₆-C₁₈ fatty alcohols (d) contained in the composition, the precondition still applies that in addition the ratio condition [(c)+(d)]/[(a)+(b)] must be satisfied.

Emulsions with especially good storage stability could be obtained if the compositions contained—in relation to their total weight—(d) one or more C₁₆-C₁₈ fatty alcohols in a total quantity of from about 0.1 to about 20.0% by weight, preferably from about 5.0 to about 15.0% by weight, more preferably from about 10.0 to about 13.0% by weight, and very particularly preferably from about 10.5 to about 12.5% by weight. Here, all values in % by weight again relate to the total quantity of the C₁₆-C₁₈ fatty alcohols (d) used in the composition, which is set in relation to the total weight of the composition.

In a further very particularly preferred embodiment a composition as contemplated herein contains—in relation to the total weight of the composition—
(d) one or more C16-C18 fatty alcohols in a total quantity of from about 0.1 to about 20.0% by weight, preferably from about 5.0 to about 15.0% by weight, more preferably from about 10.0 to about 13.0% by weight, and very particularly preferably from about 10.5 to about 12.5% by weight.
   Weight Ratio [(c)+(d)]/[(a)+(b)]

Regarding the use quantities of the emulsifier constituents (a), (b), (c) and (d), the weight ratio of the sum of all constituents [(c)+(d)] contained in the composition to the sum of all ethoxylated fatty alcohols [(a)+(b)] contained in the composition, i.e. the weight ratio [(c)+(d)]/[(a)+(b)], must lie at a value of from about 2 to about 5 as second ratio condition.

This ratio condition states that the sum of fatty acid monoglycerides (c) and C₁₆-C₁₈ fatty alcohols (d) in comparison to the sum of the highly and low-ethoxylated fatty alcohols (a) and (b) is higher by the factor from about 2 to about 5. If this ratio condition is satisfied, an O/W emulsion is formed with a mixture of non-ionic lipophilic and hydrophilic emulsifier constituents (a) to (d) which tolerates the variations in the salt content very well. Corresponding emulsions remain particularly stable at low salt contents, but in particular also at high salt contents. In addition, the viscosity of a dye cream which has a high salt content in comparison to an otherwise identical dye cream, which has a lower salt content, is only insignificant. The stability of the viscosity, independently of the salt content of the emulsion, constitutes a significant advantage in the production of a colour portfolio comprising a large number of different shades.

It has proven to be explicitly very particularly preferred if the weight ratio of the sum of all constituents [(c)+(d)] contained in the composition to the sum of all ethoxylated fatty alcohols [(c)+(d)] contained in the composition, i.e. the weight ratio [(c)+(d)], lies at a value of from about 2.0 to about 4.5, preferably from about 2.0 to about 4.0, more preferably from about 2.0 to about 3.5, and very particularly preferably from about 2.5 to about 3.0.

In a further very particularly preferred embodiment a composition as contemplated herein is therefore exemplified in that the ratio by weight of the sum of all constituents [(c)+(d)] contained in the composition to the sum of all ethoxylated fatty alcohols [(a)+(b)] contained in the composition, i.e. the weight ratio [(c)+(d)]/[(a)+(b)], lies at a value of from about 2.0 to about 4.5, preferably from about 2.0 to about 4.0, more preferably from about 2.0 to about 3.5, and very particularly preferably from about 2.5 to about 3.0.

Example: a dye cream contains, in addition to ammonia (e) and oxidation dyes (f),
(a) about 4.0% by weight of Ceteareth-50
(b) about 0.4% by weight of Ceteareth-2
(c) about 0.5% by weight of glyceryl monostearate
(d) about 11.0% by weight of cetearyl alcohol
   The weight ratio [(c)+(d)]/[(a)+(b)] is 11.5/4.4=2.6.

Example: a dye cream contains, in addition to ethanolamine (e) and oxidation dyes (f),
(a) about 4.5% by weight of Ceteth-50
(b) about 0.2% by weight of Ceteth-2
(c) about 0.7% by weight of glyceryl monostearate
(d) about 12.0% by weight of cetearyl alcohol
   The weight ratio [(c)+(d)]/[(a)+(b)] is 12.7/4.7=2.7.

### (e) Alkalising Agent

The compositions as contemplated herein are used as dye creams in the oxidative dyeing of keratin fibres, in particular hair. The oxidation dyeing of hair is performed generally at a natural, in particular alkaline pH value. For this reason, the compositions as contemplated herein are set to an alkaline pH and contain at least one alkalising agent (e).

The dye creams as contemplated herein preferably have a pH value in the range of from about 7 to about 12, preferably a pH value in the range of from about 8.0 to about 11.5. The pH values in the sense of the present disclosure are pH values that were measured at a temperature of about 22° C.

The alkalising agents usable to set the pH value are typically selected from inorganic salts, in particular of the alkali and earth alkaline metals, organic alkalising agents, in particular amines, basic amino acids and alkanolamines, and ammonia. Organic alkalising agents usable as contemplated herein are preferably selected from alkanolamines from primary, secondary or tertiary amines with a C2-C6 alkyl main body, which carries at least one hydroxyl group. Particularly preferred alkanolamines are selected from 2-aminoethan-1-ol (monoethanolamine), 2-amino-2-methylpropan-1-ol, 2-amino-2-methyl-propane-1,3-diol and triethanolamine. Inorganic alkalising agents usable as contemplated herein are preferably selected from the group formed from sodium hydroxide, potassium hydroxide, calcium hydroxide, barium hydroxide, sodium phosphate, potassium phosphate, sodium silicate, potassium silicate, sodium carbonate and potassium carbonate, preferably sodium hydroxide and/or potassium hydroxide. The basic amino acids are preferably selected from the group formed from L-arginine, D-arginine, D/L-arginine, L-lysine, D-lysine, D/L-lysine, particularly preferably L-arginine, D-arginine and D/L-arginine. Lastly, a further preferred alkalising agent is ammonia.

In a further very particularly preferred embodiment a composition as contemplated herein contains one or more alkalising agents (e) from the group of ammonia, 2-aminoethan-1-ol, 2-amino-2-methylpropan-1-ol, L-arginine, D-arginine, D/L-arginine, L-lysine, D-lysine and D/L-lysine.

### Oxidation Dye Precursors

The compositions as contemplated herein are dye creams for the oxidative dyeing of keratinous fibres, in particular human hair. The oxidative dyeing is achieved by use of at least one oxidation dye precursor (f). As further essential constituent (f), the compositions as contemplated herein therefore contain one or more oxidation dye precursors. Oxidation dye precursors can be divided into developers and couplers, wherein the developers, due to their greater sensitivity to oxygen, are usually used in the form of their physiologically acceptable colour-changing salts (for example in the form of their sulfates, hydrogen sulfates, chlorides or bromides).

Within the scope of oxidative dyeing, coupler components alone do not result in significant dyeing, and instead always require the presence of developer components. Since couplers are not as sensitive to oxygen as developers, they likewise can be used in the form of their colour-changing salts in the preparations, but are often also used in free form (i.e. not in salt form).

During the course of the works leading to this present disclosure it was found that in particular shades having a high content of oxidation dye precursors, i.e. brown shades, black shades or other dark shades, can be very well stabilised by the compositions as contemplated herein. This effect is particularly pronounced when oxidation dye precursors are used in the form of their physiologically acceptable salts.

Oxidation dye precursors of the developer type are usually derivatives of p-phenylenediamine, p-aminophenol, or heterocyclic compounds with at least one, preferably at least two amino groups. For conversion into their salts, the amino groups contained in these structures are protonated and have the corresponding equivalent of sulfate anions, hydrogen sulfate anions, chloride anions and/or bromide anions for neutralisation of this positive charge.

In the case of p-toluene diamine sulfate, this is for example the compound toluene diamine×H₂SO₄. Both amino groups are present in protonated form (in the form of ammonium ions) and the two cationic charges now contained in the molecule are neutralised by a sulfate anion (SO ²⁻). In the case of p-toluene diamine monohydrochloride, this is therefore the compound toluene diamine×HCl. One of the two amino groups is present in protonated form and has a chloride as counterion. In the case of p-toluene diamine dihydrochloride, this is the compound toluene diamine×2 HCl. Both amino groups are present in protonated form and have two chlorides as counterion. The salts of the further oxidation dyes of the developer type are composed similarly.

Preferred physiologically acceptable colour-changing salts of developers are, for example, phenylenediamine sulfate, phenylenediamine monohydrochloride, phenylenediamine dihydrochloride, p-toluene diamine sulfate, p-toluene diamine monohydrochloride, p-toluene diamine dihydrochloride, 2-(2-hydroxyethyl)-p-phenylenediamine sulfate, 2-(2-hydroxyethyl)-p-phenylenediamine monohydrochloride, 2-(2-hydroxyethyl)-p-phenylenediamine dihydrochloride, N,N-bis-(2-hydroxyethyl)-p-phenylenediamine sulfate, N,N-bis-(2-hydroxyethyl)-p-phenylenediamine monohydrochloride, N,N-bis-(2-hydroxyethyl)-p-phenylenediamine dihydrochloride, 2-methoxymethyl-p-phenylenediamine sulfate, 2-methoxymethyl-p-phenylenediamine monohydrochloride, 2-methoxymethyl-p-phenylenediamine dihydrochloride, p-aminophenol hydrogen sulfate, p-aminophenol monohydrochloride, 4-amino-3-methylphenol hydrogen sulfate, 4-amino-3-methylphenol chloride, 2,4,5,6-tetraaminopyrimidine monosulfate, 2,4,5,6-tetraaminopyrimidine disulfate, 2,4,5,6-tetraaminopyrimidine monohydrochloride, 2,4,5,6-tetraaminopyrimidine dihydrochloride, 2,4,5,6-tetraaminopyrimidine trihydrochloride, 2,4,5,6-tetraaminopyrimidine tetrahydrochloride, 4-hydroxy-2,5,6-triaminopyrimidine sulfate, 4-hydroxy-2,5,6-triaminopyrimidine monohydrochloride, 4-hydroxy-2,5,6-triaminopyrimidine diydrochloride, 4-hydroxy-2,5,6-triaminopyrimidine trihydrochloride, 4,5-diamino-1-(2-hydroxyethyl)-1H-pyrazole sulfate, 4,5-diamino-1-(2-hydroxyethyl)-1H-pyrazole monohydrochloride and/or 4,5-diamino-1-(2-hydroxyethyl)-1H-pyrazole dihydrochloride.

In a further very particularly preferred embodiment a composition as contemplated herein contains one or more oxidation dye precursors (f) from the group of phenylenediamine sulfate, phenylenediamine monohydrochloride, phenylenediamine dihydrochloride, p-toluene diamine sulfate, p-toluene diamine monohydrochloride, p-toluene diaminedihydrochloride, 2-(2-hydroxyethyl)-p-phenylenediamine sulfate, 2-(2-hydroxyethyl)-p-phenylenediamine monohydrochloride, 2-(2-hydroxyethyl)-p-phenylenediamine dihydrochloride, N,N-bis-(2-hydroxyethyl)-p-phenylenediamine sulfate, N,N-bis-(2-hydroxyethyl)-p-phenylenediamine monohydrochloride, N,N-bis-(2-hydroxyethyl)-p-phenylenediamine dihydrochloride, 2-methoxymethyl-p-phenylenediamine sulfate, 2-methoxymethyl-p-phenylenediamine monohydrochloride, 2-methoxymethyl-p-phenylenediamine dihydrochloride, p-aminophenol hydrogen sulfate, p-aminophenol monohydrochloride, 4-amino-3-methylphenol hydrogen sulfate, 4-amino-3-methylphenol chloride, 2,4,5,6-tetraaminopyrimidine monosulfate, 2,4,5,6-tetraaminopyrimidine disulfate, 2,4,5,6-tetraaminopyrimidine monohydrochloride, 2,4,5,6-tetraaminopyrimidine dihydrochloride, 2,4,5,6-tetraaminopyrimidine trihydrochloride, 2,4,5,6-tetraaminopyrimidine tetrahydrochloride, 4-hydroxy-2,5,6-triaminopyrimidine sulfate, 4-hydroxy-2,5,6-triaminopyrimidine monohydrochloride, 4-hydroxy-2,5,6-triaminopyrimidine diydrochloride, 4-hydroxy-2,5,6-triaminopyrimidine trihydrochloride, 4,5-diamino-1-(2-hydroxyethyl)-1H-pyrazole sulfate, 4,5-diamino-1-(2-hydroxyethyl)-1H-pyrazole monohydrochloride and/or 4,5-diamino-1-(2-hydroxyethyl)-1H-pyrazole dihydrochloride.

Depending on the desired colour result, oxidation dye precursors of the developer and coupler type are used in different quantities in the colouring composition.

If dyeing in a blonde shade is desired, the use of oxidation dye precursors in a total quantity below about 0.3% by weight is thus usually sufficient.

If, however, the user wishes to achieve dyeing in a very dark shade, for example in a dark-brown shade or in a black shade, this necessitates the use of oxidation dye precursors in a total quantity of at least about 2.0% by weight, often about 3.0% by weight, and in the case of particularly dark shades (black) even above about 4.5% by weight (in relation to the total weight of the composition as contemplated herein, i.e. the alkaline dye cream).

The higher is the dye content, the more difficult it is to stabilise the composition. In this regard it has been found that in particular the stabilisation of brown or black shades is very easily possible by the afore-described emulsifier system containing the constituents (a) to (d). The compositions as contemplated herein therefore preferably contain one or more oxidation dye precursors (f) from the group of sulfates, chlorides and bromides in a total quantity of from about 0.01 to about 5.5% by weight, preferably from about 0.7 to about 5.0% by weight, more preferably from about 0.9 to about 4.5% by weight, and very particularly preferably from about 2.1 to about 3.4% by weight.

In a further very particularly preferred embodiment a composition as contemplated herein contains—in relation to the total weight of the composition—one or more oxidation dye precursors (f) from the group of sulfates, chlorides and bromides in a total quantity of from about 0.01 to about 5.5% by weight, preferably from about 0.7 to about 5.0% by weight, more preferably from about 0.9 to about 4.5% by weight, and very particularly preferably from about 2.1 to about 3.4% by weight.

Oxidation dye precursors of the developer type can be contained as the sole colour-changing compounds in the composition as contemplated herein. It is preferred, however, as contemplated herein if the colouring composition (A) additionally contains at least one oxidation dye precursor of the coupler type (referred to as a coupler for short).

Within the scope of oxidative dyeing, coupler components alone do not result in significant dyeing, and instead always require the presence of developer components. Coupler components as contemplated herein allow at least one substitution of a chemical groups of the coupler by the oxidised form of the developer component. Here, covalent bonds form between the coupler and developer component.

At least one compound from one of the following classes is preferably selected as coupler component suitable as contemplated herein:

m-aminophenol and/or derivatives thereof,
m-diaminobenzene and/or derivatives thereof,
o-diaminobenzene and/or derivatives thereof,
o-aminophenol derivatives, such as o-aminophenol,
naphthalene derivatives with at least one hydroxy group,
di- or trihydroxybenzene and/or derivatives thereof,
pyridine derivatives,
pyrimidine derivatives,
monohydroxyindol derivatives and/or monoaminoindol derivatives,
monohydroxyindoline derivatives and/or monoaminoindoline derivatives,
pyrazolone derivative, such as 1-phenyl-3-methylpyrazol-5-one,
morpholine derivatives, such as 6-hydroxybenzomorpholine or 6-aminobenzomorpholine,
quinoxaline derivatives, such as 6-methyl-1,2,3,4-tetrahydroquinoxaline.
Mixtures of two or more compounds from one or more of these classes are also possible as contemplated herein within the scope of this embodiment.

A further embodiment a composition as contemplated herein contains at least one oxidation dye precursor of the coupler type, which is selected from the group of 3-aminophenol, 5-amino-2-methylphenol, 3-amino-2-chloro-6-methylphenol, 2-hydroxy-4-aminophenoxyethanol, 5-amino-4-chloro-2-methylphenol, 5-(2-hydroxyethyl)-amino-2-methylphenol, 2,4-dichloro-3-aminophenol, 2-aminophenol, 3-phenylenediamine, 2-(2,4-diaminophenoxy)ethanol, 1,3-bis(2,4-diaminophenoxy)propane, 1-methoxy-2-amino-4-(2-hydroxyethylamino)benzene, 1,3-bis(2,4-diaminophenyl)propane, 2,6-bis(2′-hydroxyethylamino)-1-methylbenzene, 2({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-({3-[1(2-hydroxyethyl)amino]-4,5-dimethylphenyl}amino)ethanol, 2-[3-morpholin-4-ylphenyl)amino]ethanol, 3-amino-4-(2-methoxy-ethoxy)-5-methylphenylamine, 1-amino-3-bis-(2-hydroxyethyl)aminobenzene, resorcinol, 2-methylresorcinol, 4-chlororesorcinol, 1,2,4-trihydroxybenzene, 2-amino-3-hydroxypyridine, 3-amino-2-methylamino-6-methoxypyridine, 2,6-dihydroxy-3,4-dimethylpyridine, 3,5-diamino-2,6-dimethoxypyridine, 1-phenyl-3-methylpyrazol-5-one, 1-naphthol, 1,5-dihydroxynaphthalene, 2,7-dihydroxynaphthalene, 1,7-dihydroxynaphthalene, 1,8-dihydroxynaphthalene, 4-hydroxyindole, 6-hydroxyindole, 7-hydroxyindole, 4-hydroxyindoline, 6-hydroxyindoline and 7-hydroxyindoline, and the physiologically acceptable colour-changing salts thereof.

In addition to the oxidation dye precursors or instead of these, the compositions as contemplated herein can contain at least one substantive dye. These are dyes that are taken up directly on the hair and do not require an oxidative process in order to form the colour. Substantive dyes are usually nitrophenylenediamines, nitroaminophenols, azo dyes, anthraquinones, triarylmethane dyes or indophenols.

Substantive dyes can be divided into anionic, cationic and non-ionic substantive dyes.

In particular, non-ionic nitro and quinone dyes and neutral azo dyes are suitable as non-ionic substantive dyes. Preferred non-ionic substantive dyes are the compounds known under the following international names or trade names: HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9, and 1,4-diamino-2-nitrobenzene, 2-amino-4-nitrophenol, 1,4-bis-(2-hydroxyethyl)-amino-2-nitrobenzene, 3-nitro-4-(2-hydroxyethyl)-aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)-amino-5-chloro-2-nitrobenzene, 4-amino-3-nitrophenol, 1-(2′-ureidoethyl)amino-4-nitrobenzene, 2-[(4-amino-2-nitrophenyl)amino]-benzoic acid, 6-nitro-1,2,3,4-tetrahydroquinoxaline, 2-hydroxy-1,4-naphthoquinone, picramic acid and colour-changing salts thereof, 2-amino-6-chloro-4-nitrophenol, 4-ethylamino-3-nitrobenzoic acid and 2-chloro-6-ethylamino-4-nitrophenol.

Anionic substantive dyes carry at least one negative charge and are also referred to in the literature as acid dyes. Preferred anionic substantive dyes are the compounds known under the international names or trade names: bromophenol blue, tetrabromophenol blue, Acid Yellow 1, Yellow 10, Acid Yellow 23, Acid Yellow 36, Acid Orange 7, Acid Red 33, Acid Red 52, Pigment Red 57:1, Acid Blue 7, Acid Green 50, Acid Violet 43, Acid Black 1 and Acid Black 52.

Cationic dyes are exemplified by the presence of at least one positive charge. In the English literature, cationic dyes are also referred to as “basic dyes”. Preferred cationic substantive dyes are Basic Blue 7, Basic Blue 26, Basic Violet 2 and Basic Violet 14, Basic Yellow 57, Basic Red 76, Basic Blue 16, Basic Blue 347 (Cationic Blue 347/Dystar), HC Blue No. 16, Basic Blue 99, Basic Brown 16, Basic Brown 17, Yellow 87, Basic Orange 31 and Basic Red 51.

As already described beforehand, the stabilisation of emulsions is often more difficult, the higher is the salt content of an emulsion. Emulsions in which high quantities of cationic substantive dyes are used are also usually more difficult to stabilise than emulsions having a low dye content or emulsions that contain only non-ionic substantive dyes.

During the course of the works performed, it was found that the colouring compositions present in emulsion form in particular can be stabilised very well also if the composition contains additionally a cationic and/or anionic substantive dye.

In principle, the substantive dyes can be contained in a total quantity from about 0.001 to about 10% by weight in the composition as contemplated herein.

Within the scope of a further embodiment, a composition for dyeing keratinous fibres that has proven to be very particularly preferred is one which contains, in an aqueous carrier,
(a) one or more highly-ethoxylated C₁₆-C₁₈ fatty alcohols of formula

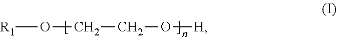

in which
R1 represents a linear or branched, saturated or unsaturated C₁₆-C₁₈ alkyl group and
n represents an integer from about 35 to about 90,
b) one or more low-ethoxylated C₁₆-C₁₈ fatty alcohols of formula (II),

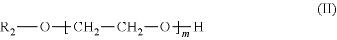

in which
R2 represents a linear or branched, saturated or unsaturated C16-C18 alkyl group and
m represents an integer from about 1 to about 4.
(c) one or more monoesters of glycerol and a C14-C20 fatty acid,
(d) one or more C16-C18 fatty alcohols,
(e) one or more alkalising agents, and
(f) one or more oxidation dye precursors,
   wherein
   the weight ratio of all highly ethoxylated C₁₆-C₁₈ fatty alcohols (a) contained in the composition to all low-ethoxylated C₁₆-C₁₈ fatty alcohols (b) contained in the composition, i.e. the weight ratio (a)/(b), lies at a value of from about 10 to about 20, and
   the weight ratio of the sum of all constituents [(c)+(d)] contained in the composition to the sum of all ethoxylated fatty alcohols [(a)+(b)] contained in the composition, i.e. the weight ratio [(c)+(d)]/[(a)+(b)], lies at a value of from about 2 to about 5.

Within the scope of a further embodiment, a composition for dyeing keratinous fibres that has proven to be very particularly preferred is one which contains, in an aqueous carrier,
(a) one or more highly-ethoxylated C₁₆-C₁₈ fatty alcohols of formula (I)

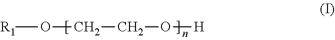

in which
R1 represents a linear or branched, saturated or unsaturated C16-C18 alkyl group and
n represents an integer from about 40 to about 80,
b) one or more low-ethoxylated C₁₆-C₁₈ fatty alcohols of formula (II),

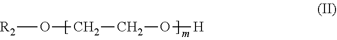

in which
R2 represents a linear or branched, saturated or unsaturated C16-C18 alkyl group and
m represents an integer from about 1 to about 3.
(c) one or more monoesters of glycerol and a C14-C20 fatty acid,
(d) one or more C16-C18 fatty alcohols,
(e) one or more alkalising agents, and
(f) one or more oxidation dye precursors,
   wherein
   the weight ratio of all highly ethoxylated C16-C18 fatty alcohols (a) contained in the composition to all low-ethoxylated C16-C18 fatty alcohols (b) contained in the composition, i.e. the weight ratio (a)/(b), lies at a value of from about 10 to about 20, and
   the weight ratio of the sum of all constituents [(c)+(d)] contained in the composition to the sum of all ethoxylated fatty alcohols [(a)+(b)] contained in the composition, i.e. the weight ratio [(c)+(d)]/[(a)+(b)], lies at a value of from about 2 to about 5.

Within the scope of a further embodiment, a composition for dyeing keratinous fibres that has proven to be very particularly preferred is one which contains, in an aqueous carrier,
(a) one or more highly-ethoxylated C₁₆-C₁₈ fatty alcohols of formula (I)

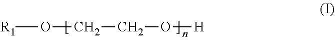

in which
R1 represents a linear or branched, saturated or unsaturated C16-C18 alkyl group and
n represents an integer from about 45 to about 70,
b) one or more low-ethoxylated C₁₆-C₁₈ fatty alcohols of formula (II),

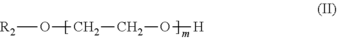

in which
R2 represents a linear or branched, saturated or unsaturated C16-C18 alkyl group and
m represents an integer from about 1 to about 3.
(c) one or more monoesters of glycerol and a C14-C20 fatty acid,
(d) one or more C16-C18 fatty alcohols,
(e) one or more alkalising agents, and
(f) one or more oxidation dye precursors,
   wherein
   the weight ratio of all highly ethoxylated C16-C18 fatty alcohols (a) contained in the composition to all low-ethoxylated C16-C18 fatty alcohols (b) contained in the composition, i.e. the weight ratio (a)/(b), lies at a value of from about 10 to about 20, and
   the weight ratio of the sum of all constituents [(c)+(d)] contained in the composition to the sum of all ethoxylated fatty alcohols [(a)+(b)] contained in the composition, i.e. the weight ratio [(c)+(d)]/[(a)+(b)], lies at a value of from about 2 to about 5.

Within the scope of a further embodiment, a composition for dyeing keratinous fibres that has proven to be very particularly preferred is one which contains, in an aqueous carrier,
(a) one or more highly ethoxylated C₁₆-C₁₈ fatty alcohols with from about 30 to about 100 ethoxy groups,
(b) one or more low-ethoxylated C₁₆-C₁₈ fatty alcohols with 1 to 5 ethoxy groups,
(c) one or more monoesters of glycerol and a C₁₄-C₂₀ fatty acid,
(d) one or more C₁₆-C₁₈ fatty alcohols,
(e) one or more alkalising agents, and
(f) one or more oxidation dye precursors,
   wherein
   the weight ratio of all highly ethoxylated C₁₆-C₁₈ fatty alcohols (a) contained in the composition to all low-ethoxylated C₁₆-C₁₈ fatty alcohols (b) contained in the composition, i.e. the weight ratio (a)/(b), lies at a value of from about 10 to about 20, and
   the weight ratio of the sum of all constituents [(c)+(d)] contained in the composition to the sum of all ethoxylated fatty alcohols [(a)+(b)] contained in the composition, i.e. the weight ratio [(c)+(d)]/[(a)+(b)], lies at a value of from about 2 to about 5,
   and wherein the composition contains—in relation to its total weight—
   one or more highly ethoxylated C₁₆-C₁₈ fatty alcohols (a) with from about 30 to about 100 ethoxy groups in a total quantity of from about 2.0 to about 10.0% by weight,
   one or more low-ethoxylated C₁₆-C₁₈ fatty alcohols (b) with 1 to 5 ethoxy groups in a total quantity of from about 0.05 to about 1.5% by weight,
   one or more monoesters of glycerol and a C₁₄-C₂₀ fatty acid (c) in a total quantity of from about 0.1 to about 5.0% by weight, and
   one or more C₁₆-C₁₈ fatty alcohols (d) in a total quantity of from about 0.1 to about 20.0% by weight.

Within the scope of a further embodiment, a composition for dyeing keratinous fibres that has proven to be very particularly preferred is one which contains, in an aqueous carrier,
(a) one or more highly ethoxylated C₁₆-C₁₈ fatty alcohols with from about 30 to about 100 ethoxy groups,
(b) one or more low-ethoxylated C₁₆-C₁₈ fatty alcohols with 1 to 5 ethoxy groups,
(c) one or more monoesters of glycerol and a C₁₄-C₂₀ fatty acid,
(d) one or more C₁₆-C₁₈ fatty alcohols,
(e) one or more alkalising agents, and
(f) one or more oxidation dye precursors,
   wherein
   the weight ratio of all highly ethoxylated C₁₆-C₁₈ fatty alcohols (a) contained in the composition to all low-ethoxylated C₁₆-C₁₈ fatty alcohols (b) contained in the composition, i.e. the weight ratio (a)/(b), lies at a value of from about 10 to about 20, and
   the weight ratio of the sum of all constituents [(c)+(d)] contained in the composition to the sum of all ethoxylated fatty alcohols [(a)+(b)] contained in the composition, i.e. the weight ratio [(c)+(d)]/[(a)+(b)], lies at a value of from about 2 to about 5,
   and wherein the composition contains—in relation to its total weight—
   one or more highly ethoxylated C₁₆-C₁₈ fatty alcohols (a) with from about 30 to about 100 ethoxy groups in a total quantity of from about 2.0 to about 8.0% by weight,
   one or more low-ethoxylated C₁₆-C₁₈ fatty alcohols (b) with 1 to 5 ethoxy groups in a total quantity of from about 0.05 to about 1.25% by weight,
   one or more monoesters of glycerol and a C₁₄-C₂₀ fatty acid (c) in a total quantity of from about 0.1 to about 2.5% by weight, and
   one or more C₁₆-C₁₈ fatty alcohols (d) in a total quantity of from about 5.0 to about 15.0% by weight.

Within the scope of a further embodiment, a composition for dyeing keratinous fibres that has proven to be very particularly preferred is one which contains, in an aqueous carrier,
(a) one or more highly ethoxylated C₁₆-C₁₈ fatty alcohols with from about 30 to about 100 ethoxy groups,
(b) one or more low-ethoxylated C₁₆-C₁₈ fatty alcohols with 1 to 5 ethoxy groups,
(c) one or more monoesters of glycerol and a C₁₄-C₂₀ fatty acid,
(d) one or more C₁₆-C₁₈ fatty alcohols,
(e) one or more alkalising agents, and
(f) one or more oxidation dye precursors,
   wherein
   the weight ratio of all highly ethoxylated C₁₆-C₁₈ fatty alcohols (a) contained in the composition to all low-ethoxylated C₁₆-C₁₈ fatty alcohols (b) contained in the composition, i.e. the weight ratio (a)/(b), lies at a value of from about 10 to about 20, and
   the weight ratio of the sum of all constituents [(c)+(d)] contained in the composition to the sum of all ethoxylated fatty alcohols [(a)+(b)] contained in the composition, i.e. the weight ratio [(c)+(d)]/[(a)+(b)], lies at a value of from about 2 to about 5, and wherein the composition contains—in relation to its total weight—
   one or more highly ethoxylated C₁₆-C₁₈ fatty alcohols (a) with from about 30 to about 100 ethoxy groups in a total quantity of from about 2.0 to about 6.0% by weight,
   one or more low-ethoxylated C₁₆-C₁₈ fatty alcohols (b) with 1 to 5 ethoxy groups in a total quantity of from about 0.1 to about 1.0% by weight,
   one or more monoesters of glycerol and a C₁₄-C₂₀ fatty acid (c) in a total quantity of from about 0.1 to about 1.3% by weight, and
   one or more C₁₆-C₁₈ fatty alcohols (d) in a total quantity of from about 10.0 to about 13.0% by weight.

Within the scope of a further embodiment, a composition for dyeing keratinous fibres that has proven to be very particularly preferred is one which contains, in an aqueous carrier,
(a) one or more highly ethoxylated C₁₆-C₁₈ fatty alcohols with from about 30 to about 100 ethoxy groups,
(b) one or more low-ethoxylated C₁₆-C₁₈ fatty alcohols with 1 to 5 ethoxy groups,
(c) one or more monoesters of glycerol and a C₁₄-C₂₀ fatty acid,
(d) one or more C₁₆-C₁₈ fatty alcohols,
(e) one or more alkalising agents, and
(f) one or more oxidation dye precursors,
   wherein
   the weight ratio of all highly ethoxylated C₁₆-C₁₈ fatty alcohols (a) contained in the composition to all low-ethoxylated C₁₆-C₁₈ fatty alcohols (b) contained in the composition, i.e. the weight ratio (a)/(b), lies at a value of from about 10 to about 20, and
   the weight ratio of the sum of all constituents [(c)+(d)] contained in the composition to the sum of all ethoxylated fatty alcohols [(a)+(b)] contained in the composition, i.e. the weight ratio [(c)+(d)]/[(a)+(b)], lies at a value of from about 2 to about 5, and wherein the composition contains—in relation to its total weight—
   one or more highly ethoxylated C₁₆-C₁₈ fatty alcohols (a) with from about 30 to about 100 ethoxy groups in a total quantity of from about 3.0 to about 5.0% by weight,
   one or more low-ethoxylated C₁₆-C₁₈ fatty alcohols (b) with 1 to 5 ethoxy groups in a total quantity of from about 0.1 to about 0.75% by weight,
   one or more monoesters of glycerol and a C₁₄-C₂₀ fatty acid (c) in a total quantity of from about 0.2 to about 0.7% by weight, and
   one or more C₁₆-C₁₈ fatty alcohols (d) in a total quantity of from about 10.5 to about 12.5% by weight.

Within the scope of a further embodiment, a composition for dyeing keratinous fibres that has proven to be very particularly preferred is one which contains, in an aqueous carrier,
(a) one or more highly ethoxylated C₁₆-C₁₈ fatty alcohols with from about 30 to about 100 ethoxy groups,
(b) one or more low-ethoxylated C₁₆-C₁₈ fatty alcohols with 1 to 5 ethoxy groups,
(c) one or more monoesters of glycerol and a C₁₄-C₂₀ fatty acid,
(d) one or more C₁₆-C₁₈ fatty alcohols,
(e) one or more alkalising agents, and
(f) one or more oxidation dye precursors,
   wherein
   the weight ratio of all highly ethoxylated C₁₆-C₁₈ fatty alcohols (a) contained in the composition to all low-ethoxylated C₁₆-C₁₈ fatty alcohols (b) contained in the composition, i.e. the weight ratio (a)/(b), lies at a value of from about 11 to about 19, and
   the weight ratio of the sum of all constituents [(c)+(d)] contained in the composition to the sum of all ethoxylated fatty alcohols [(a)+(b)] contained in the composition, i.e. the weight ratio [(c)+(d)]/[(a)+(b)], lies at a value of from about 2 to about 4.5.

Within the scope of a further embodiment, a composition for dyeing keratinous fibres that has proven to be very particularly preferred is one which contains, in an aqueous carrier,
(a) one or more highly ethoxylated C₁₆-C₁₈ fatty alcohols with from about 30 to about 100 ethoxy groups,
(b) one or more low-ethoxylated C₁₆-C₁₈ fatty alcohols with 1 to 5 ethoxy groups,
(c) one or more monoesters of glycerol and a C₁₄-C₂₀ fatty acid,
(d) one or more C₁₆-C₁₈ fatty alcohols,
(e) one or more alkalising agents, and
(f) one or more oxidation dye precursors,
   wherein
   the weight ratio of all highly ethoxylated C₁₆-C₁₈ fatty alcohols (a) contained in the composition to all low-ethoxylated C₁₆-C₁₈ fatty alcohols (b) contained in the composition, i.e. the weight ratio (a)/(b), lies at a value of from about 12 to about 18, and
   the weight ratio of the sum of all constituents [(c)+(d)] contained in the composition to the sum of all ethoxylated fatty alcohols [(a)+(b)] contained in the composition, i.e. the weight ratio [(c)+(d)]/[(a)+(b)], lies at a value of from about 2.0 to about 4.0.

Within the scope of a further embodiment, a composition for dyeing keratinous fibres that has proven to be very particularly preferred is one which contains, in an aqueous carrier,
(a) one or more highly ethoxylated C₁₆-C₁₈ fatty alcohols with from about 30 to about 100 ethoxy groups,
(b) one or more low-ethoxylated C₁₆-C₁₈ fatty alcohols with 1 to 5 ethoxy groups,
(c) one or more monoesters of glycerol and a C₁₄-C₂₀ fatty acid,
(d) one or more C₁₆-C₁₈ fatty alcohols,
(e) one or more alkalising agents, and
(f) one or more oxidation dye precursors,
   wherein
   the weight ratio of all highly ethoxylated C₁₆-C₁₈ fatty alcohols (a) contained in the composition to all low-ethoxylated C₁₆-C₁₈ fatty alcohols (b) contained in the composition, i.e. the weight ratio (a)/(b), lies at a value of from about 11 to about 19, preferably from about 13 to about 17, and
   the weight ratio of the sum of all constituents [(c)+(d)] contained in the composition to the sum of all ethoxylated fatty alcohols [(a)+(b)] contained in the composition, i.e. the weight ratio [(c)+(d)]/[(a)⇄(b)], lies at a value of from about 2.0 to about 3.5.

Within the scope of a further embodiment, a composition for dyeing keratinous fibres that has proven to be very particularly preferred is one which contains, in an aqueous carrier,
(a) one or more highly ethoxylated C₁₆-C₁₈ fatty alcohols with from about 30 to about 100 ethoxy groups,
(b) one or more low-ethoxylated C₁₆-C₁₈ fatty alcohols with 1 to 5 ethoxy groups,
(c) one or more monoesters of glycerol and a C₁₄-C₂₀ fatty acid,
(d) one or more C₁₆-C₁₈ is fatty alcohols,
(e) one or more alkalising agents, and
(f) one or more oxidation dye precursors,
   wherein
   the weight ratio of all highly ethoxylated C₁₆-C₁₈ fatty alcohols (a) contained in the composition to all low-ethoxylated C₁₆-C₁₈ fatty alcohols (b) contained in the composition, i.e. the weight ratio (a)/(b), lies at a value of from about 14 to about 16, and
   the weight ratio of the sum of all constituents [(c)+(d)] contained in the composition to the sum of all ethoxylated fatty alcohols [(a)+(b)] contained in the composition, i.e. the weight ratio [(c)+(d)]/[(a)+(b)], lies at a value of from about 2.5 to about 3.0.

### Viscosity

As previously described, a particularly stable emulsion which also has excellent viscosity stability can be produced by the use of the non-ionic emulsifiers (a) to (d) in dye creams which contain alkalising agents (e) and oxidation dye precursors (f). In this context, “viscosity stability” means that the emulsion reacts to changes in the salt content only with minimal viscosity fluctuations.

The viscosities were measured in the work leading to this present disclosure using a viscometer of the Haake Rheostress 6000 type at about 20° C. (about 20° C./Haake Rheostress 6000/measured with cone 35/1 geometry, diameter about 35 mm and about 1° angle/shear rate about 7.2 s-1).

By use of the constituents (a) to (d) in the previously described quantity ranges and ratios, it could be ensured that the viscosities of the dye creams were in the range of from about 10,000 to about 30,000 mPas, preferably from about 12,000 to about 25,000 mPas, and very particularly preferably from about 16,000 to about 21,000 mPas (about 20° C./Haake Rheostress 6000/measured with cone 35/1 geometry, diameter about 35 mm and about 1° angle/shear rate about 7.2 s-1).

In a further very particularly preferred embodiment a composition as contemplated herein has a viscosity of from about 10,000 to about 30,000 mPas, preferably from about 12,000 to about 25,000 mPas, and very particularly preferably from about 16,000 to about 21,000 mPas (about 20° C./Haake Rheostress 6000/measured with cone 35/1 geometry, diameter about 35 mm and about 1° angle/shear rate about 7.2 s-1).

### Branched Fatty Alcohols

Within the scope of a further embodiment it may prove to be advantageous if the composition as contemplated herein additionally contains the branched fatty alcohols 2-octyldodecanol (i.e. a branched C₂₀ fatty alcohol).

The quantity of the 2-octyldodecanol used in the composition lies here—in relation to the total weight of the composition—preferably in a range of from about 0.1 to about 10% by weight, preferably from about 0.5 to about 5.0% by weight, and very particularly preferably from about 1.0 to about 3.0% by weight.

In a further very particularly preferred embodiment a composition as contemplated herein contains—in relation to the total weight of the composition—from about 0.1 to about 10% by weight, preferably from about 0.5 to about 5.0% by weight, and very particularly preferably from about 1.0 to about 3.0% by weight of octyldodecanol.

Multi-component Packaging Unit (Kit-of-parts)

The compositions as contemplated herein are compositions for oxidative changing of the colour of keratinous fibres. In particular, they are compositions for the dyeing, especially oxidative dyeing, of human hair. In order to initiate the oxidative dyeing process and to form the oxidation dyes, the dye cream is mixed with an oxidant preparation just before use. In this way the ready-to-use oxidative dye which is applied to the user's hair is produced.

In order to avoid incompatibilities and in order to prevent a premature, undesirable dye formation, the dye cream (referred to hereinafter as agent (A)) and the oxidant preparation necessary for the oxidative colouring (agent (B)) are always packaged separately from one another and are brought into contact with one another just before use. For the consumer, the two components are provided preferably in the form of a multi-component packaging unit (kit-of-parts).

A second subject of the present disclosure is therefore a multi-component packaging unit (kit-of-parts) for the oxidative dyeing of keratinous fibres, in particular human hair, comprising, packaged separately from one another,

a container (I) containing a cosmetic agent (A) and
a container (II) containing a cosmetic agent (B), wherein
the agent (A) in container (I) is an agent as has been disclosed in detail in the description of the first subject of the present disclosure, and
the agent (B) in the container (II) is an oxidant preparation (B) containing hydrogen peroxide.

### Oxidant Preparation (B)

The oxidant preparation (B) contains hydrogen peroxide as oxidant. The hydrogen peroxide can be provided either as hydrogen peroxide itself or also in the form of its solid addition products with organic or inorganic compounds, such as urea, melamine and sodium borate.

The quantity of oxidant in the oxidant preparation (B)—in relation to the total weight of the oxidant preparation (B)—is preferably from about 0.5 to about 12% by weight, preferably from about 2 to about 10% by weight, particularly preferably from about 3 to about 6% by weight (calculated as about 100% H202).

Such oxidant preparations are preferably aqueous, flowable oxidant preparations. Here, preferred preparations are exemplified in that the flowable oxidant preparation—in relation to its weight—contains from about 40 to about 90% by weight, preferably from about 50 to about 85% by weight, particularly preferably from about 55 to about 85% by weight, more preferably from about 60 to about 85% by weight, and in particular from about 70 to about 85% by weight of water.

It has also proven to be advantageous if the oxidant preparation (B) contains at least one stabiliser or complexing agent. Conventional complexing agents and stabilisers that are preferred within the scope of the present disclosure are, for example polyoxycarboxylic acids, polyamines, ethylenediaminetetraacetic acid (EDTA), N-hydroxyethylethylenediaminetriacetic acid, diethylenetriaminepentaacetic acid (DTPA), ethylenediaminedisuccinic acid (EDDS), hydroxyethyliminodiacetic acid, nitridodiacetic acid-3-propionic acid, isoserindiacetic acid, N,N-di-(2-hydroxyethyl)glycine, N-(1,2-dicarboxy-2-hydroxyethyl)glycine, N-(1,2-dicarboxy-2-hydroxyethyl)aspartic acid or nitrilotriacetic acid (NTA), ethylenediaminediglutaric acid (EDGA), 2-hydroxypropylendiamindisuccinc acid (HPDS), glycinamide-N,N′-disuccinic acid (GADS), ethylenediamine-N-N′-diglutaric acid (EDDG), 2-hydroxypropylenediamine-N-N′-disuccinic acid (HPDDS), diaminoalkyldi(sulfosuccinic acid) (DDS), ethylenedicysteic acid (EDC), ethylenediamine-N-N′-bis(ortho-hydroxyphenyl)acetic acid (EDDHA), N-2-hydroxyethylamine-N,N-diacetic acid, glyceryliminodiacetic acid, iminodiacetic acid-N-2-hydroxypropylsulfonic acid, aspartic acid-N-carboxymethyl-N-2,5-hydroxypropyl-3-sulfonic acid, β-alanine-N,N′-diacetic acid, aspartic acid-N,N′-diacetic acid, aspartic acid-N-monoacetic acid, dipicolinic acid, and colour-changing salts and/or derivatives thereof, geminal diphosphonic acids such as 1-hydroxyethane-1,1-diphosphonic acid (HEDP), higher homologues thereof with up to 8 carbon atoms and hydroxy or amino group-containing derivatives hereof and 1-aminoethane-1,1-diphosphonic acid, higher homologues thereof with up to 8 carbon atoms and hydroxy or amino group-containing derivatives, aminophosphonic acids such as ethylenediaminetetra(methylenephosphonic acid) (EDTMP), diethylene-triaminepenta(methylenephosphonic acid) (DTPMP) and higher homologues thereof, or nitrilotri(methylenephosphonic acid), phosphonopolycarboxylic acids such as 2-phosphonobutan-1,2,4-tricarboxylic acid, cyclodextrins, and alkalistannates (sodium stannate), alkalipyrophosphates (tetrasodiumpyrophosphate, disodiumpyrophosphate), alkaliphosphates (sodiumphosphate), and phosphoric acid and colour-changing salts thereof.

### Further Ingredients

The agent (A) as contemplated herein and/or the oxidant preparation (B) may additionally contain further active substances, auxiliaries and additives which are different from the previously described constituents (a) to (f). These may be, for example: cationic surfactants, amphoteric surfactants, anionic surfactants, non-ionic surfactants (which are different from constituents (a) to (d)), anionic, non-ionic and/or cationic polymers, structurants such as glucose, maleic acid and lactic acid, perfume oils, fibre-structure-improving active substances, in particular mono-, di-and oligosaccharides such as glucose, galactose, fructose, fruit sugar and lactose; dyes for staining the composition; anti-dandruff active substances such as piroctone olamine, zinc omadine and climbazole; amino acids and oligopeptides; protein hydrolysates on an animal and/or vegetable basis, and in the form of their fatty acid condensation products or optionally anionically or cationically modified derivatives; vegetable oils; light stabilisers and UV blockers; active substances such as panthenol, pantothenic acid, pantolactone, allantoin, pyrrolidinone carboxylic acids and their colour-changing salts and bisabolol; polyphenols, in particular hydroxycinnamic acids, 6,7-dihydroxycoumarins, hydroxybenzoic acids, catechins, tannins, leucoanthocyanidins, anthocyanidins, flavanones, flavones and flavonols; ceramides or pseudoceramides; vitamins, provitamins and vitamin precursors; plant extracts; swelling and penetration substances such as glycerol, propylene glycol monoethyl ether, carbonates, hydrogen carbonates, guanidines, ureas and primary, secondary and tertiary phosphates; opacifiers such as latex, styrene/PVP and styrene/acrylamide copolymers; pearlescent agents such as ethylene glycol mono- and distearate and PEG-3 distearate; pigments and propellants such as propane-butane mixtures, N2O, dimethyl ether, CO2 and air.

The additional active substances and auxiliaries are used in the compositions as contemplated herein preferably in quantities of, in each case, from about 0.0001 to about 10% by weight, in particular from about 0.0005 to about 5% by weight, in relation to the total weight of the agent (A) or the oxidant preparation (B).

That said in respect of the composition as contemplated herein applies mutatis mutandis to the preferred embodiments of the multi-component packaging unit as contemplated herein.

### EXAMPLES

The following compositions were produced (all values in % by weight)

| | Ex 1 | Ex 2 | Ex. 3 |
|---|---|---|---|
| Ceteareth-50 | 3.70 | 3.70 | 3.70 |
| Ceteareth-2 | 0.24 | 0.24 | 0.24 |
| Glyceryl monostearate | 0.50 | 0.50 | 0.50 |
| Cetearyl alcohol | 11.0 | 11.0 | 11.0 |
| Ammonia (25% aqueous solution) | 6.00 | 6.00 | 6.00 |
| Titanium dioxide | 0.5 | 0.5 | 0.5 |
| Monoethanol amine | 0.6 | 0.6 | 0.6 |
| Sodium sulfite | 0.35 | 0.35 | 0.35 |
| Linoleamidopropyl PG-Dimoniumchloride, | 0.03 | 0.03 | 0.03 |
| Phosphate | | | |
| EDTA (disodium salt) | 0.2 | 0.2 | 0.2 |
| Polyquatemium-39 (dimethyldiallylammonium | 0.15 | 0.15 | 0.15 |
| chloride, acrylamide, acrylic acid terpolymer) | | | |
| Vitamin C | 0.1 | 0.1 | 0.1 |
| p-toluene diamine, sulfate | 1.178 | 0.011 | 0.22 |
| Resorcinol | 0.275 | — | 0.03 |
| 2-methylresorcinol | 0.266 | — | 0.1 |
| m-aminophenol | 0.0378 | — | — |
| 2-amino-3-hydroxypryridine | 0.038 | — | 0.19 |
| 2-amino-4-(hydroxyethylamino)anisole, sulfate | — | 0.01 | |
| 5-amino-2-methylphenol | — | 0.001 | 0.45 |
| 4-amino-3-methylphenol | — | — | 0.37 |
| Water | to 100 | to 100 | to 100 |
| Viscosity (20° C./Haake Rheostress 6000/ | 20094 mPas | 20161 mPas | 19204 mPas |
| measured with Cone 35/1 geometry, diameter | | | |
| 35 mm and 1° angle/shear rate 7.2 s−1) 2 months | | | |
| after production | | | |

| | Ex 4 | Ex 5 | Ex 6 |
|---|---|---|---|
| Ceteareth-50 | 3.70 | 3.70 | 3.70 |
| Ceteareth-2 | 0.24 | 0.24 | 0.24 |
| Glyceryl monostearate | 0.50 | 0.50 | 0.50 |
| Cetearyl alcohol | 11.0 | 11.0 | 11.0 |
| Ammonia (25% aqueous solution) | 6.00 | 6.00 | 6.00 |
| Titanium dioxide | 0.5 | 0.5 | 0.5 |
| Monoethanol amine | 0.6 | 0.6 | 0.6 |
| Sodium sulfite | 0.35 | 0.35 | 0.35 |
| Linoleamidopropyl PG-Dimoniumchloride, | 0.03 | 0.03 | 0.03 |
| Phosphate | | | |
| EDTA (disodium salt) | 0.2 | 0.2 | 0.2 |
| Polyquatemium-39 (dimethyldiallylammonium | 0.15 | 0.15 | 0.15 |
| chloride, acrylamide, acrylic acid terpolymer) | | | |
| Vitamin C | 0.1 | 0.1 | 0.1 |
| p-toluene diamine, sulfate | 0.85 | 2.55 | 0.295 |
| Resorcinol | 0.26 | 0.90 | 0.13 |
| 2-methylresorcinol | — | | 0.05 |
| m-aminophenol | 0.10 | 0.33 | 0.01 |
| 2-amino-3-hydroxypryridine | — | — | — |
| 2-amino-4-(hydroxyethylamino)anisole, sulfate | — | 0.03 | — |
| 5-amino-2-methylphenol | — | — | — |
| 4-chlororesorcinol | 9.13 | — | — |
| 2,4-diaminophenoxyethanol, dihydrochloride | — | — | — |
| 4-amino-3-methylphenol | — | — | 0.05 |
| Water | to 100 | to 100 | to 100 |
| Viscosity (20° C./Haake Rheostress 6000/ | 20897 mPas | 19234 mPas | 20584 mPas |
| measured with Cone 35/1 geometry, diameter | | | |
| 35 mm and 1° angle/shear rate 7.2 s−1) 2 months | | | |
| after production | | | |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A composition for dyeing keratinous fibres, the composition comprising, in an aqueous carrier,
(a) one or more highly ethoxylated C₁₆-C₁₈ fatty alcohols with from about 30 to about 100 ethoxy groups,
(b) one or more low-ethoxylated C₁₆-C₁₈ fatty alcohols with from about 1 to about 5 ethoxy groups,
(c) optionally one or more monoesters of glycerol and a C₁₄-C₂₀ fatty acid,
(d) one or more C₁₆-C₁₈ fatty alcohols,
(e) one or more alkalising agents, and
(f) one or more oxidation dye precursors,
wherein
the weight ratio of all highly ethoxylated C₁₆-C₁₈ fatty alcohols (a) to all low-ethoxylated C₁₆-C₁₈ fatty alcohols (b) is from about 10 to about 20, and
the weight ratio of the sum of all constituents [(c)+(d)] to the sum of all ethoxylated fatty alcohols [(a)+(b)] is from about 2 to about 5.

2. The composition according to claim 1, wherein the (a) one or more highly ethoxylated C₁₆-C₁₈ fatty alcohols with from about 30 to about 100 ethoxy groups are of formula (I), 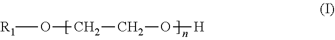
in which
R1 represents a linear or branched, saturated or unsaturated C₁₆-C₁₈ alkyl group and
n represents an integer from about 30 to about 100.

3. The composition according to claim 1, wherein the composition comprises—in relation to the total weight of the composition—
(a) one or more highly ethoxylated C₁₆-C₁₈ fatty alcohols with from about 30 to about 100 ethoxy groups in a total quantity of from about 2.0 to about 10.0% by weight.

4. The composition according to claims 1, wherein the composition comprises (b) one or more low-ethoxylated C₁₆-C₁₈ fatty alcohols of formula (II), 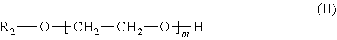
in which
R2 represents a linear or branched, saturated or unsaturated C₁₆-C₁₈ alkyl group and
m represents an integer from about 1 to about 5.

5. The composition according to claim 1, wherein the composition comprises—in relation to the total weight of the composition—
(b) one or more low-ethoxylated C₁₆-C₁₈ fatty alcohols with from about 1 to about 5 ethoxy groups in a total quantity of from about 0.05 to about 1.5% by weight.

6. The composition according to claim 1, wherein the weight ratio of all highly ethoxylated C₁₆-C₁₈ fatty alcohols (a) to all low-ethoxylated C₁₆-C₁₈ fatty alcohols (b) is from about 11 to about 19.

7. The composition according to claim 1, wherein the composition comprises (c) one or more monoesters of glycerol and a C₁₄-C₂₀ fatty acid of formula (III) 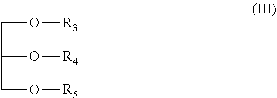
in which
R3, R4, R5, independently of one another, represent a hydrogen atom or an acyl group —C(O)R₆, in which R₆ represents an unbranched or branched, saturated or unsaturated C₁₃-C₁₉ alkyl group.

8. The composition according to claim 1, wherein the composition comprises—in relation to the total weight of the composition—
(c) one or more monoesters from glycerol and a C₁₄-C₂₀ fatty acid in a total quantity from about 0.1 to about 5.0% by weight.

9. The composition according to claim 1, wherein the composition comprises—in relation to the total weight of the composition—
(d) one or more C₁₆-C₁₈ fatty alcohols in a total quantity of from about 0.1 to about 20.0% by weight.

10. The composition according to claim 1, wherein the weight ratio of the sum of all constituents [(c)+(d)] to the sum of all ethoxylated fatty alcohols [(a)+(b)] is from about 2.0 to about 4.5.

11. The composition according to claim 1, wherein the composition comprises one or more alkalising agents (e) from the group of ammonia, 2-aminoethan-1-ol, 2-amino-2-methylpropan-1-ol, 2-amino-2-methyl-propane-1,3-diol, L-arginine, D-arginine, D/L-arginine, L-lysine, D-lysine and D/L-lysine.

12. The composition according to claim 1, wherein the composition comprises one or more oxidation dye precursors (f) from the group of phenylenediamine sulfate, phenylenediamine monohydrochloride, phenylenediamine dihydrochloride, p-toluene diamine sulfate, p-toluene diamine monohydrochloride, p-toluene diamine dihydrochloride, 2-(2-hydroxyethyl)-p-phenylenediamine sulfate, 2-(2-hydroxyethyl)-p-phenylenediamine monohydrochloride, 2-(2-hydroxyethyl)-p-phenylenediamine dihydrochloride, N,N-bis-(2-hydroxyethyl)-p-phenylenediamine sulfate, N,N-bis-(2-hydroxyethyl)-p-phenylenediamine monohydrochloride, N,N-bis-(2-hydroxyethyl)-p-phenylenediamine dihydrochloride, 2-methoxymethyl-p-phenylenediamine sulfate, 2-methoxymethyl-p-phenylenediamine monohydrochloride, 2-methoxymethyl-p-phenylenediamine dihydrochloride, p-aminophenol hydrogen sulfate, p-aminophenol monohydrochloride, 4-amino-3-methylphenol hydrogen sulfate, 4-amino-3-methylphenol chloride, 2,4,5,6-tetraaminopyrimidine monosulfate, 2,4,5,6-tetraaminopyrimidine disulfate, 2,4,5,6-tetraaminopyrimidine monohydrochloride, 2,4,5,6-tetraaminopyrimidine dihydrochloride, 2,4,5,6-tetraaminopyrimidine trihydrochloride, 2,4,5,6-tetraaminopyrimidine tetrahydrochloride, 4-hydroxy-2,5,6-triaminopyrimidine sulfate, 4-hydroxy-2,5,6-triaminopyrimidine monohydrochloride, 4-hydroxy-2,5,6-triaminopyrimidine dihydrochloride, 4-hydroxy-2,5,6-triaminopyrimidine trihydrochloride, 4,5-diamino-1-(2-hydroxyethyl)-1H-pyrazole sulfate, 4,5-diamino-1-(2-hydroxyethyl)-1H-pyrazole monohydrochloride and/or 4,5-diamino-1-(2-hydroxyethyl)-1H-pyrazole dihydrochloride.

13. The composition according to claim 1, wherein the composition comprises—in relation to the total weight of the composition—one or more oxidation dye precursors (f) from the group of sulfates, chlorides and bromides in a total quantity of from about 0.01 to about 5.5% by weight.

14. The composition according to claim 1, wherein the composition comprises has a viscosity of from about 10,000 to about 30,000 mPas (20° C./Haake Rheostress 6000/measured with cone 35/1 geometry, diameter 35 mm and 1° angle/shear rate 7.2 s⁻¹).

15. The composition according to claim 1, wherein the composition comprises—in relation to the total weight of the composition—from about 0.1 to about 10% by weight of octyldodecanol.

16. A multi-component packaging unit (kit-of-parts) for the oxidative dyeing of keratin fibres comprising, packaged separately from one another,
a container (I) comprising a cosmetic agent (A) and
a container (II) comprising a cosmetic agent (B),
wherein
the agent (A) is the composition for dyeing keratinous fibres of claim 1,
the agent (B) is an oxidant preparation comprising hydrogen peroxide.
